(19) United States
(12) Patent Application Publication
(10) Pub. No.: US 2010/0098748 A1
(43) Pub. Date: Apr. 22, 2010

(54) Arg-Gly-Asp (RGD) Sequence Containing Cyclic Peptide and Its Active Targeting Liposomes

(76) Inventors: Jiyao Wang, Shanghai (CN); Shilin Du, Shanghai (CN); Weiyne Lu, Shanghai (CN)

Correspondence Address: WARE FRESSOLA VAN DER SLUYS & ADOLPHSON, LLP, BRADFORD GREEN, BUILDING 5, 755 MAIN STREET, P O BOX 224, MONROE, CT, 06468, US

(21) Appl. No.: 11/990,577

(22) PCT Filed: Aug. 15, 2005
(86) PCT No.: PCT/CN2005/001258

§ 371 Date: Feb. 14, 2008

(30) Foreign Application Priority Data
Mar. 25, 2005 (CN) 200510024656.8

(51) Int. Cl. A61K 9/127; C07K 5/12; A61K 38/21

(52) U.S. Cl. 424/450; 530/317; 424/85.4

(57) ABSTRACT

The present invention relates to the field of pharmaceutics and clinical pharmacy, involving the preparation method and application of a cyclic peptide containing Argnine-Glycine-Aspartate sequence and its active targeting liposomes. The cyclic peptide described here meets the requirement for ligand in that it forms ring by amide linkage with stable sterical structure, it shows low propensity to degrade, and it carries an active hydrosulfide group which is easy to modify carriers. The artificially synthesized peptide, which has a small molecular weight and low propensity to cause immune response, can be used as the ligand to bind with the integrin on the surface of HSC. The active targeting liposome established with the cyclic peptide realized cell-targeted therapy of experimental hepatic fibrosis via receptor-mediated pathway. The invention can target RGD cyclic peptide-labeled interferon-loaded liposomes to the fibrotic liver and its good efficacy in the treatment of hepatic fibrosis has been proven by experiments in rats in vitro and in vivo.

### CROSS REFERENCE TO RELATED APPLICATIONS

The present application claims priority to International Application Number PCT/CN2005/001258, filed on Aug. 15, 2005, which is hereby incorporation by reference in its entirety.

### TECHNICAL FIELD

The present invention relates to the field of pharmaceutics and clinical pharmacy, involving a polypeptide containing Arg-Gly-Asp (RGD) sequence, which is a ligand of integrin, and its cyclic formation as well as the establishment and medical application of receptor-mediated active targeted liposome, which is targeted to hepatic stellate cells (HSC). Specially, the preparation and application in the treatment of liver fibrosis of the cyclic peptide containing Arg-Gly-Asp (RGD) sequence and its active targeting liposome are involved.

### BACKGROUND

The liver fibrosis of the liver is the common pathologic basis of all liver diseases and the earlier and unavoidable period of the development of hepatic cirrhosis. According to statistical data, 25%˜40% of liver fibrosis eventually developed into hepatic cirrhosis. The pathologic changes can be caused by different etiological factors. For example, after causing chronic liver injury, factors such as virus, alcohol and parasites will activate hepatic stellate cells (HSC), induce the increased synthesis, the decreased degradation or the decompensations of extracellular matrix (ECM) mainly composed of collagen, and then lead to the abnormal sedimentation of extracellular matrix in the liver, resulting in hepatic cirrhosis. Studies showed that hyperplasia and activation of HSCs are the cytological basis of the genesis of liver cirrhosis and the common key element of the formation of liver cirrhosis caused by various etiological factors (Frieman S. L. Semin Liver Dis. 1990, 10(1): 20-29). Therefore the therapy targeted to HSC is possible to reverse liver fibrosis. Because HSCs are located in the interspaces surrounding the liver sinus and only take a small proportion (approximately 5%), it is difficult to design specific therapy targeted to HSC.

Targeting drug delivery systems can selectively concentrate the drug at the action site with the aid of carriers to improve the efficacy of drug and decrease the toxicity and side effects, especially for cytotoxic drug. Successful targeting formulations should have the following three characteristics, site-specific accumulation, controlled drug release, and non-toxicity and biodegradability. According to the presence of the active group on the liposome surface, the targeting mechanism of liposomes can be divided into passive targeting and active targeting. Passive targeting liposome which carries no active groups can be enriched at certain organs or focus by means of the physiological characteristics and differences of various organs in human body, whereas active targeting liposome can be targeted to specific cells by affinity between cells and active mediating groups such as ligands and monoclonal antibodies which have been introduced onto the liposome surface. In comparison to passive targeting, active targeting has better specificity, thus elevating liposome targeting from focus/organ level to cell level. Active targeting liposome which can exhibit controlled-release in vivo in theory is the best approach for pharmaceutical research to increase drug efficacy and decrease toxicity.

In 1990, Klibanov et al (Klibanov A L, et al. FEBS Lett, 1990, 268(2): 235; Bume G, Biochim Biophys Acta, 1990, 1029(1): 91) reported a long-circulating liposome the membrane of which contained palmitoyl glucosiduronate or polyethyleneglycol (PEG)-conjuated lipids (such as PEG-DSPE), which was usually called sterically stabilized liposomes (SSL) or long circulating liposomes (LCL). Because these liposomes have highly hydrated hydrophilic groups on their surface, the binding of many components, especially opsonin, to the liposomes are blocked, thus inhibiting the phagocytosis of mononuclear phagocyte system (MPS). Because SSL can sustain the release of drug and enhance the selectivity to specific target tissue, it is applicable in many aspects. Studies (Lasic D D, et al, Biochim Biophys Acta, 1991, 1070(1):187) showed that SSL is superior to classic liposome (CL) in that it can prolong the retention time in the circulation, deduce the rate and extent of the phagocytosis of mononuclear phagocyte system, increase absorption of target sites such as tumor tissue and infection tissue, possess the ability to permeate the biological barrier and exhibit dose-independence and thus linear pharmacokinetics in animals and human.

Integrin is a membrane surface glycoprotein receptor family, mainly mediating the adhension of cells to extracellular matrix (ECM) which is the ligand of integrin. Integrin is composed of two sub-units (α and β). Different integrin composed of different combination of the two subunits has different ECM as its ligand and exhibits different function. Arg-Gly-Asp (RGD) tri-peptide sequence is the common recognition site of integrin. The surface of HSC can express different integrins. Static HSCs only express α-1 and rarely other subunits but activated HSCs express many integrins.

To date, no HSC-targeted integrin-mediated targeting liposome has been reported for the treatment of hepatic fibrosis.

### DISCLOSURE OF THE INVENTION

The invention provides a cyclic peptide containing Arg-Gly-Asp (RGD) sequence and active targeting liposomes which are modified by the above-mentioned cyclic peptide, loaded with drug such as interferon and actively targeted to the integrin on the surface of hepatic stellate cell for the treatment of hepatic fibrosis. This invention further provides methods for preparation of the above-mentioned cyclic peptide and active targeting liposomes.

The RGD cyclic peptide described in the invention is an oligopeptide composed of eight amino acid residues with the sequence Cys-Gly-Arg-Gly-Asp-Trp-Pro-Lys (C*GRGDSPK*), in which * denotes the position of cyclization and the RGD sequence is the binding site of integrin on the surface of HSCs.

The above-mentioned RGD cyclic peptide forms ring by the amide linkage between cysteine and lysine residue with free hydrosulfide group on the cysteine terminal.

The amino acid sequence of the above-mentioned RGD cyclic peptide may be X*GRGDSPZ*, in which * denotes the cyclization position, X denotes cysteine residue containing a free hydrosulfide group and Z denotes any amino acid which can form ring with cysteine residue.

The amino acid sequence of the above-mentioned RGD cyclic peptide may be X*YRGDYZ*, in which * denotes the cyclization position, X denotes cysteine residue containing a free hydrosulfide group, Y denotes at least one or a sequence of any length composed of the following amino acids which would not influence the binding with target receptors including alanie, argnine, asparagine, aspartate, glumatic acid, glycine, histidine, isoleucine, leucine, lysine, methionine, phenylalanine, praline, serine, threonine, tyrosine, valine, and Z denotes any amino acid which can cyclized with c cysteine residue.

As is shown by the following reaction function, the free hydrosulfide group on the RGD cyclic peptide may be conjugated to maleoyl-polyethylene glycol lipid derivative (MAL-PEG-DOPE), thus connecting the RGD cyclic peptide to the liposome surface. MAL-PEG-DOPE described here is biodegradable which is mainly excreted by the kidney.

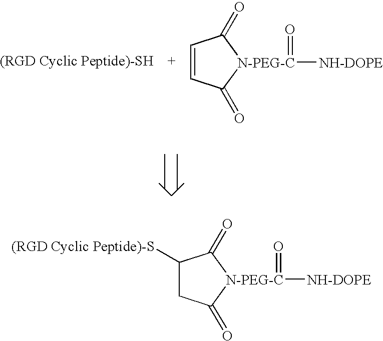

The artificial synthesized cyclic peptide containing RGD sequence provided in this invention has the following advantages: 1. Arg-Gly-Asp (RGD) sequence contained in RGD cyclic peptide is the specific binding site of integrin on the surface of HSC, characteristic of exogenous ligand in that it binds with HSC with high specificity, time and concentration-dependence, saturability and competitive inhibition; 2. The amide linkage (—CO—NH—) which cyclized the RGD cyclic peptide is stable in the sterical conformation and unlikely to degrade; 3. The active hydrosulfide group (—SH) in the cysteine residue in RGD cyclic peptide is easy to further modify carriers; 4. As an artificial functional peptide with a small molecular weight, RGD cyclic peptide is less likely to induce immune reaction.

The liposomes described in the invention are prepared by rotary evaporation-thin film hydration-extrusion method. The liposomes described in the invention include classical liposomes (CL), sterically stabilized liposomes (SSL), RGD cyclic peptide modified classical liposomes (RGD-CL), RGD cyclic peptide modified sterically stabilized liposomes (RGD-SSL) and interferon encapsulating-classical liposomes (IFN-CL), sterically stabilized liposomes (IFN-SSL), RGD cyclic peptide modified classical liposomes (IFN-ROD-CL), RGD cyclic peptide-modified sterically stabilized liposomes (IFN-ROD-SSL).

The membrane material of CL described in the invention is composed of egg phospholipid (EPC), cholesterol (Chol), maleoyl-polyethylene glycol lipid derivatives (MAL-PEG₃₃₄₀-DOPE) with the molar ration 2:1:0.02.

The membrane material of SSL described in the invention is composed of egg phospholipid (EPC), cholesterol (Chol), monooxymethyl-polyethylene glycol lipid derivative and MAL-PEG₃₃₄₀-DOPE with the molar ration 2:1:0.1:0.02 and PEG content 3.2 mol %.

RGD cyclic peptide can be connected to the surface of liposomes by adding RGD cyclic peptide into the membrane material for the preparation of CL and SSL with the molar ratio of MAL-PEG-DOPE: RGD cyclic peptide 10:1 and covalently conjugated to MAL-PEG-DOPE by the hydrosulfide group. The unconjugated RGD cyclic peptide was removed by gel column chromatography to obtain RGD-CL or RGD-SSL.

The invention further added interferon solution (IFN) into CL, SSL, RGD-CL or RGD-SSL, vortexed the mixture for 30 min in ice bath, and then removed unencapsulated IFN by gel column chromatography. The encapsulation efficiency of IFN is 35.6% and the drug loading is 10⁴ U IFN/umol lipid.

Extrusion method was used to homogenize the particle size of CL, SSL, RGD-CL, RGD-SSL, CL-IFN, SSL-IFN, RGD-CL-IFN and RGD-SSL-IFN, obtaining liposomes with the particle size range of 50˜200 nm and the optimal size of 100 nm.

The active targeting liposomes targeted at the integrin on the surface of HSC provided in the invention target the treatment to experimental hepatic fibrosis via the receptor-mediated pathway. The anti-fibrotic efficacy was achieved by targeting RGD cyclic peptide-labeled interferon-loaded actively-targeted liposomes to the fibrotic liver by means of specific interaction between the surface receptor of HSC and the artificial RGD cyclic peptide.

The experiment in vitro was showed by the fluorescence tracing method that the active targeting liposomes could specifically bind with HSC which were separated from rats.

In the experiment in vivo, SPET imaging showed that RGD-SSL mainly distributed in liver for 24 hr and were basically excreted via the kidney. The hepatic fibrotic rat model was established by ligation of the common bile duct. After administration into the candal vein, the therapeutic efficacy of RGD-SSL-IFN on the hepatic fibrotic rat was observed. Results showed that liver function, serous hepatic-fibrotic index, hydroxyproline content of liver tissue and hepatic pathological changes were significantly improved for RGD-SSL-IFN group in comparison with SSL-IFN group. The expression of hepatic type I collagen mRNA and α-actin of HSC was significantly decreased, indicating that this active targeting liposomes had good therapeutic effect on hepatic fibrosis in rats.

### BRIEF DESCRIPTION OF THE FIGURES

FIG. 1 shows the histological staining of liver tissue for various groups of rats including HE staining (×100), hepatic fibrotic group (A), interferon-liposome group (C), RGD cyclic peptide-liposome-interferon (E), Desmin staining (×400), hepatic fibrotic group (B). interferon-liposome group (D), RGD cyclic peptide-liposome-interferon (F).

FIG. 2 shows liver function for various groups of rats including 1. sham operation group 2. hepatic fibrotic model group 3. interferon-liposome group 4. RGD cyclic peptide-liposome-interferon group. Compared with the sham operation group, ΔP<0.05; compared with the BDL group, *P<0.05; compared with the BDL-IFN-SSL group, ▴P<0.05. (ALT, alanine aminotransferase; AST, aspirate transferase; ALP, alkaline phosphate, TBIL, total bilirubin; γ-GT, glutamyl transferase).

FIG. 3 shows serous hepatic fibrotic indexes for various groups of rats including 1. sham operation group 2. hepatic fibrotic model group 3. interferon-liposome group 4. RGD cyclic peptide-liposome-interferon group. Compared with the sham operation group, ΔP<0.05; compared with the BDL group, *P<0.05; compared with the BDL-IFN-SSL group, ▴P<0.05. (HA, hyaluronic acid, PCIII: III type precollagen, LN, laminin, C_{IV}: IV type collagen)

FIG. 4 shows hydroxyproline (HYP) content in liver tissue in various groups of rats (mg/g liver tissue) including 1. sham operation group 2. hepatic fibrotic model group 3. interferon-liposome group 4. RGD cyclic peptide-liposome-interferon group. Compared with the sham operation group, ΔP<0.05; compared with the BDL group, *P<0.05; compared with the BDL-IFN-SSL group, ▴P<0.05.

FIG. 5 shows the expression of collagen I mRNA in liver tissue for various groups of rats including 1. sham operation group 2. hepatic fibrotic model group 3. interferon-liposome group 4. RGD cyclic peptide-liposome-interferon group. A. collagen I mRNA eletrophoresis strip. B. collagen I mRNA'GAPDH mRNA relative gray scale. Compared with the sham operation group, ΔP<0.05; compared with the BDL group, *P<0.05; compared with the BDL-IFN-SSL group, ▴P<0.05.

FIG. 6 shows the expression of α-actin (α-SMA) in liver tissue for various groups of rats including 1. blank control group 2. sham operation group 3. hepatic fibrotic model group 4. interferon-liposome group 5. RGD cyclic peptide-liposome-interferon group.

### MODES FOR CARRYING OUT THE INVENTION

### Example 1

### Preparation of RGD Cyclic Peptide

According to the method reported by literature (Schnolzer M, Alewood P, Jones A, Alewood D, Kent S B. Int J Pept Protein Res. 1992, 40(3-4):180-93), cysteine—Glycine-Argine-Glycine-Asparate-Tryptophan-Proline-Lysine thioester PAM (CysGlyArgGlyAspSerProLys-SCH2CO-leu-PAM) resin was synthesized by protein synthesizer. What was different from literature was that Boc-amino acid (2.2 mmol) was activated in N,N-dimethylformamide (DMF) containing the condensing agent (HBTU 2.0 mmol) and N,N-diisopropyl ethylamine (DIEA 20%, v/v) for 3 min and then added into the resin (0.25 mol) to react for 10 min. N-Boc protecting groups were removed by trifluoroacetic acid (TFA). In the whole process of synthesis, DMF and dichlormethane (DCM) was used to wash the resin. The protecting groups of these used amino acid were Arg(Tosyl), Asp (OcHxl), Cys (4MeBzl), Lys(2ClZ), Ser(Bzl). After the synthesis, the resin was stirred in anhydrous hydrofluoric acid containing 5% p-cresol at 0° C. for 1 hr. The crude product was precipitated by cold ethyl ether and then purified by high-pressure preparative chromatography. The purified sample was dissolved in 0.25M phosphate buffer (pH 7.5) containing 6M GuHCl and then 2% thiophenol was added. The solution was purified and lyophilized to obtain RGD cyclic peptide. The obtained RGD cyclic peptide reacted with fluorescein isothiocyanate in phosphate buffer (pH 9.0) to obtain FITC-RGD after further purification and lyophilization. The purity was above 95% according to HPLC identification.

### Example 2

### Preparation of Liposomes

Rotary evaporation-thin file hydration-extrusion method was used according to following procedures:
1 Egg phospholipid (EPC), cholesterol (Chol), monooxymethyl-polyethylene glycol lipid derivatives and MAL-PEG₂₀₀₀-DOPE were accurately weighed according to the molar ratio 2:1:0.1:0.02, dissolved in chloroform and rotary-evaporated at 40° C. to evaporate the organic solvent to form transparent thin film. Then phosphate buffer (PBS, pH 7.4, 22° C.) was added to fully hydrate the thin film. Homogeneous sterically stabilized liposomes (SSL) were obtained by repeated extrution through 100 nm filter membrane with Mini Extruber for 15 times. The content of PEG was 3.2mol %.
EPC, Chol and MAL-PEG₃₄₅₀-DOPE with the molar ratio being 2:1:0.02 were accurately weighed, dissolved in chloroform and rotary-evaporated at 40° C. to evaporate the organic solvent and form transparent thin film. Then phosphate buffer (PBS, pH 7.4, 22° C.) was added to fully hydrate the thin film. Homogeneous classical liposomes (CL) were obtained by repeated extrution through 100 nm filter membrane with Mini Extruber for 15 times.
RGD cyclic peptide was added into CL or SSL PBS solution according to the molar ratio RGD: MAL-PEG-DOPE 10:1 and then vibrated overnight at room temperature (25° C.). Unconjugated RGD cyclic peptide was removed by gel column chromatography (CL-4B) to obtain RGD-CL or RGD-SSL.
IFN solution (IFN-α1b) to be encapsulated was added into CL, SSL, RDG-CL, RGD-SSL and then vortex for 30 min in ice bath. Unencapsulated IFN was removed by gel column chromatography (CL-4B) to obtain CL-IFN, SSL-IFN, RGD-CL-IFN or RGD-SSL-IFN.
In the preparation of CL, SSL, RGD-CL or RGD-SSL, calcein liposomes were also prepared by replacing PBS by calcein (CF) solution (50 mmol/L) as the hydrating solution. The encapsulation efficiency of CF was measured by spectrofluorometry to be 10% (λₑₓ=492 nm, λₑₘ=512 nm).
2 Study of the shape and size of particles: A small quantity of SSL and RGD-SSL was diluted and negatively stained by 1% phosphotungstic acid for observation under transmission electron microscope. It was observed that the liposome shape showed no change after modification and encapsulation of interferon and remained homogeneous, exhibiting the typical fingerprint structure. The average particle size of liposomes was measure by laser scattering to be 98.1±23.1 nm.
Determination of encapsulation efficiency: Encapsulation efficiency of interferon was measured by enzyme linked immunosorbent assay (ELISA) to be 10⁴ U IFN/μ mol phospholipid. The activity of interferon was maintained after encapsulation by the virus inhibition method.
3 Other characteristics: Five batches of RGD-SSL-IFN were selected. pH value, was measured to be 7.21±0.08. RGD-SSL-IFN was negatively charged according to cellulose acetate membrane eletrophoresis (experimental conditions: the upper and lower electrode liquid 0.01mol/L, pH 7.4 phosphate buffer, voltage 200V). The relative viscosity was measured to be 1.0323×10⁻³±3.7460×10⁻⁵ Pa·s⁻¹ by the Ubbelohde viscometer. The relative density was measured to be 1.0025±1.37×10⁻⁴ by the pycnometric method.

### Example 3

### Investigation of Feasibility of Artificially Synthesized Cyclic Peptide Containing RGD Sequence as the Ligand of Integrin by the Fluorescence and Radioactive Isotope Tracer Method

According to the feature of ligand-receptor binding, i.e. specificity, concentration and time-dependence, competitive inhibition, fluorescein isothiocyanate (FITC)-labeled RGD cyclic peptide was co-incubated with HSC. Results showed that the binding characteristics of RGD cyclic peptide and HSC followed the basic feature of ligand-receptor binding. Equilibration dissociation constant (W) and the binding site number of each cell (Bmax) was measured by Scatchard analysis of the radioactive ligand of ³H labeled RGD cyclic peptide to be 7.05×10⁻⁹ mmol/L and 6.79×10⁵, respectively.

The procedures were as follows,

1. HSC were separated from rats.

2. Investigation of the binding of activated HSC and cyclic peptide by the fluorescence tracer method.

HSC were inoculated on 6-well plate and cultivated overnight in 0.25% FBS-DMEM after adherence. The plate was blocked by 1% BSA-DMEM before experiment. The relative fluorescence intensity of HSC bound was measured with flow cytometry.

2.1 Concentration-efficacy relationship: Cyclic peptide of different concentrations was added into each well and cultivated for 4 hr at 4° C. and 37° C., respectively.

2.2 Time-efficacy relationship: 200 nmol/L cyclic peptide was added into each well and cultivated for 0˜8 hr at 4° C. and 37° C., respectively

2.3 Competitive inhibition experiment: Unlabelled cyclic peptide at different concentration was added into each well. And then FITC-labelled cyclic peptide (200 nmol/L) was added for further binding for 4 hr.

3. Determination of equilibration dissociation constant (Kd) between RGD cyclic peptide and HSC and the number of binding sites for each cell (Bmax) by binding analysis of radioactive ligand

Hyzone-labeled cRGD(³H-RGD) was used. 0.5 mL cell suspension and 0.1 mL ³H-RGD with different concentration were placed in the reaction tube, made up the volume to 1 mL and incubated at 4° C. for 3 hr. Ice cold Hank's solution was added to stop the reaction. After centrifugation, the supernatant was removed and the sediment was water-bathed with formic acid to determine the radioactive count. Samples of each level consisted of three tubes. Total binding (TB) and non-specific binding (NSB) was established. Equilibration dissociation constant (Kd) between ³H-RGD and HSC and the maximum binding site number of each cell (Bmax) were calculated according to Scatchard model.

### Example 4

### Characterization of the Binding of RGD Cyclic Peptide Modified Liposomes and HSC by Fluorescence Tracing Method

RGD cyclic peptide-modified calcein-encapsulated liposomes (RGD-SSL-CF) and calcein-encapsulated liposomes (SSL-CF) were prepared according to example 2. RGD-SSL-CF and SSL-CF were co-cultured with HSC for 4 hr. Results showed that HSC uptook RGD-SSL-CF 5.4 times as much as SSL-CF. RGD-SSL-CF was able to specifically bind with HSC.

The steps were as follows,

1. HSC were separated from rats and cultured.

2. Investigation of the binding of activated HSC and calcein-encapsulated RGD cyclic peptide-modified liposomes (RGD-SSL-CF) by fluorescence tracer method.

HSC were plated on 33 cm² culture dish and cultured overnight in 0.25% FBS-DMEM after adherence. The plate was blocked by 1% BSA-DMEM before experiment. RGD-SSL-CF and SSL-CF were added and co-cultured for 4 hr, respectively. Then the cells were erased and dissolved in PBS (1% Triton X-100). The fluorescence intensity bound by the cells were determined by fluorospectrophotometer (Ex=492 nm, Em=512 nm). The binding of RGD-SSL-CF and HSC was observed by fluorescence microscope.

### Example 5

### ^{99m}Tc Labeled Liposomes

Liposomes were prepared according to Example 2 by adding DTPA-DOPE and phospholipids at the molar ratio of 1:10. ^{99m}Tc was labeled onto RGD-cyclic peptide-liposome by SnCl₂ reduction method. The following procedures were followed: SnCl₂ was dissolved in 0.15mol/L HCl to prepare 10 ug/uL SnCl₂—HCl solution. 100 ug SnCl₂ and then 2 mCi 99 mTcO₄ was added into 0.5 mL liposome. After mixing, the mixture was placed at room temperature for 15 min.

### Example 6

### The Distribution and Imaging In Vivo of ^{99m}Tc Labeled RGD-Cyclic Peptide-Modified Liposome in Normal and Hepatic Fibrotic Rats

After injection via candal vein, the distribution and SPET imaging in vivo of ^{99m}Tc labeled RGD-cyclic peptide-modified liposomes was studied in normal and hepatic fibrotic rats. Results showed that RGD-SSL mainly concentrated in the liver for 24 hr and excreted by the kidney.

The following procedures were followed:

0.5 mL ^{99m}Tc—RGD-SSL and ^{99m}Tc—SSL (2 mCi) were injected into the candal vein of normal and hepatic fibrotic rats. Single photon emission computed tomography (SPECT) scanning was performed at different time points with Philis-IRIX tri-probe SPECT. The distance between the probe and the rat was maintained at 2 cm and the positive image was collected with the collecting matrix being 512×512, 50 frames/sec. The interested area of each organ was drawn and the total count/interested area (counts/pixel) was measured as the counts/pixel for each organ.

### Example 7

### Therapeutic Efficacy of RGD Cyclic Peptide Modified Interferon-Loaded Liposome on the Hepatic Fibrotic Rats

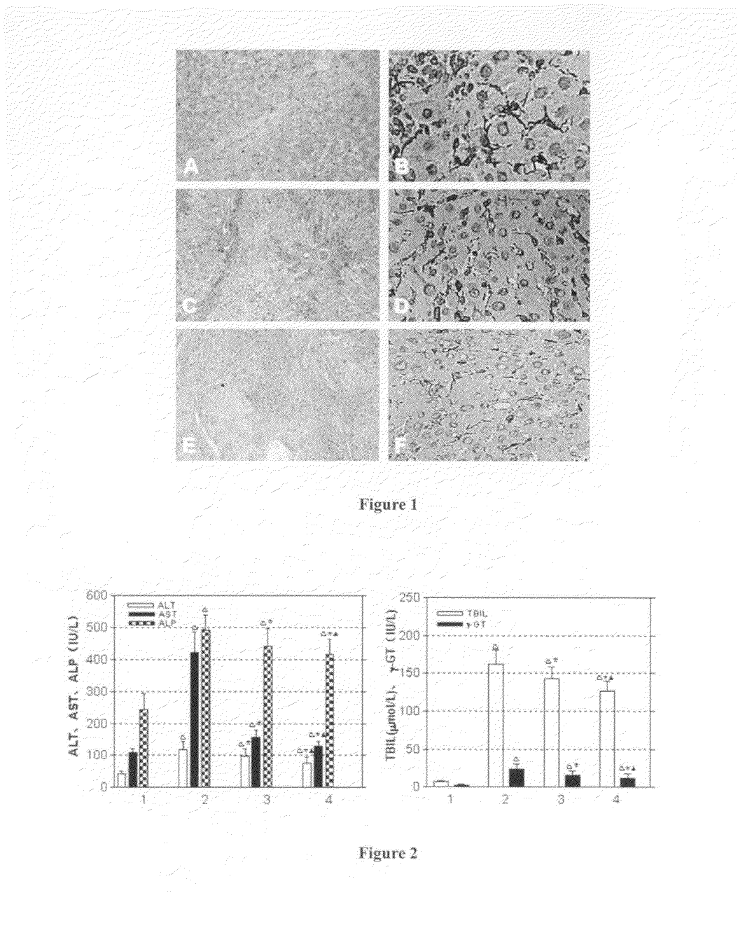

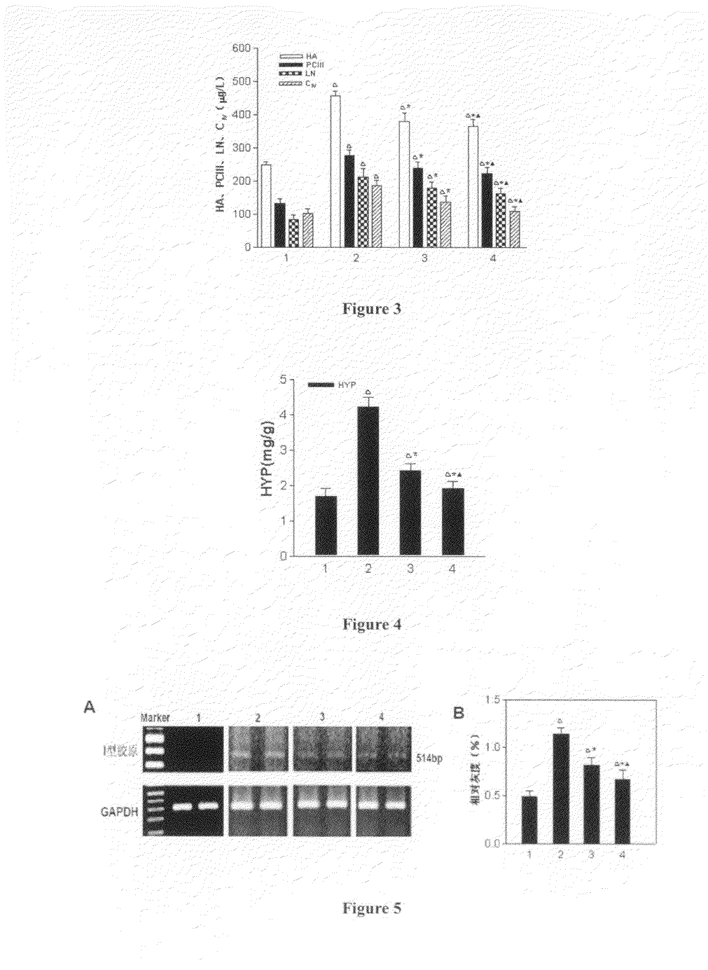

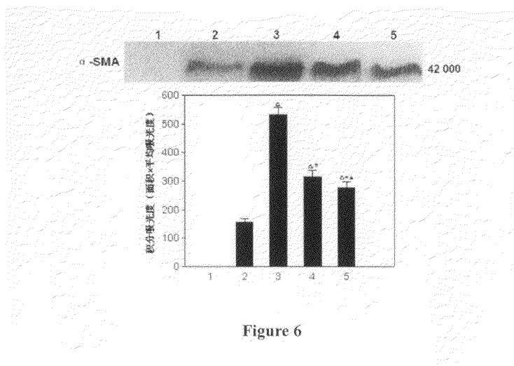

The therapeutic efficacy of RGD cyclic peptide-modified interferon-loaded liposome (RGD-SSL-IFN) on the hepatic fibrotic rat was observed after injection via the candal vein. Results showed that hepatic function, serous hepatic fibrotic indexes, content of hydroproline in liver tissue and hepatic pathological changes for the treatment group was significantly improved in comparison with interferon-loaded liposome (SSL-IFN) and that expression of hepatic I type collagen mRNA and α-actin in HSC was significantly decreased (FIGS. 1-6).

The procedures were as follows:

1. Preparation of animal models and grouping

Rats were randomized into four 4 groups including the sham operation group, the model group (BDL), the IFN-SSL (BDL+IFN-SSL) group and the interferon-liposome treatment group (BDL+IFN-RGD-SSL). Each group consisted of 10 rats. Except for the sham operation group, the common bile tract was double ligated and sheared for all rats of the other three groups. For the sham operation group, the common bile tract was exposed and separated after the abdomen was cut open, and then the abdomen was closed. From the day of ligation, 0.2 mL IFN-SSL was injected via the candal vein once a week at the dose of 5×10⁴ U IFN for the BDL+IFN-SSL group, 0.2 mL IFN-RGD-SSL was injected via the candal vein once a week at the dose of 5×10⁴ U IFN for the BDL+IFN-RGD-SSL group, and normal saline of the same volume was injected for the BDL group and the sham operation group. After continuous experiment for 4 weeks, animals were sacrificed 24 hr after the last administration to get serum and liver tissue.

2. Indexes to be observed, measurements to be conducted and methods

2.1 Observation of the general state and jaundice of rats

2.2 Observation of immunohistolochemical staining for HE and α-SMA and pathological changes

2.3 Determination of serous alanine aminotransferase (ALT), aspartate aminotransferase (AST), total bilirubin (TBIL), alkaline phosphatase (AKP) and γ-GT(γ-glutamine) with automatic biochemistry analyzer

2.4 Determination of content of serous hyaluronic acid (HA), III type precollagen(PCIII), laminin (LN) and IV type collagen (C_{IV}) with radioimmunoassay

2.5 Determination of content of hydroproline (Hyp) in the homogenate of liver tissue expressed as the content of Hyp in each gram of liver tissue with colorimetry.

2.6 Determination of expression of collagen I mRNA in the liver tissue with RT-PCR

Total RNA was extracted from the liver tissue and cDNA was synthesized. Coamplification and quantitative PCR was conducted and the product of amplification was quantified with gel system. The relative content of collagen mRNA was expressed as the ratio of collagen I absorbance×area to GAPDH absorbance×area.

2.7 Determination of expression of HSC a-actin with Western blot

HSC were separated from rat liver with two-step collagenase perfusion and density gradient centrifugation. Proteins were extracted, and the concentration of protein was determined. Degenerated SDS-polyacrylamide gel electrophoresis was performed. After electrotransfer, the nylon membrane was blocked with 5% defatted milk powder, incubated with the primary antibody (rabbit-anti-mouse antibody) and then with horseradish peroxidase labeled secondary antibody (donkey-anti-rabbit antibody). After exposure and developing, the gray scale of the specific strip on Western blot was analyzed with graphic analytical software provided by Biomad Co. The strength of the specific strip was expressed by the cumulative absorbance to perform semi-quantitative comparison of expression of the target protein. What is claimed is:

1. A cyclic peptide containing Argnine-Glycine-Aspartate sequence, characterized by the presence of Argnine-Glycine-Aspartate sequence, a cyclized amide linkage (—CO—NH—) and an active hydrosulfide group at a cysteine terminal.

2. The cyclic peptide containing Argnine-Glycine-Aspartate sequence of claim 1, wherein the amino acid sequence of the cyclic peptide is X*YRGDYZ*, in which * denotes the cyclization position, X denotes a cysteine residue containing a free hydrosulfide group, Y denotes at least one amino acid or an amino acid sequence of an adequate length, and Z denotes any amino acid which can form a ring with the cysteine residue.

3. The cyclic peptide containing Argnine-Glycine-Aspartate sequence of claim 1, wherein the amino acid sequence of the cyclic peptide is X*GRGDSPZ*, in which * denotes the cyclization position, X denotes a cysteine residue, containing a free hydrosulfide group, and Z denotes at least one amino acid or an amino acid sequence of an adequate length.

4. The cyclic peptide containing Argnine-Glycine-Aspartate sequence of claim 1, wherein the amino acid sequence of the cyclic peptide is X*GRGDSPK*, in which * denotes the cyclization position, X denotes a cysteine residue containing a free hydrosulfide group.

5. The cyclic peptide containing Argnine-Glycine-Aspartate sequence claim 1, wherein it is used in preparation of exogenous ligands of integrin on the surface of hepatic stellate cells.

6. An active targeting liposome composed of the cyclic peptide containing Argnin-Glycine-Aspartate sequence (RGD) of claim 1, wherein said active targeting liposome is prepared by covalently conjugating the active hydrosulfide group of the cysteine residue of the RGD cyclic peptide to a maleimide in a membrane material of liposomes as shown in the following reaction:

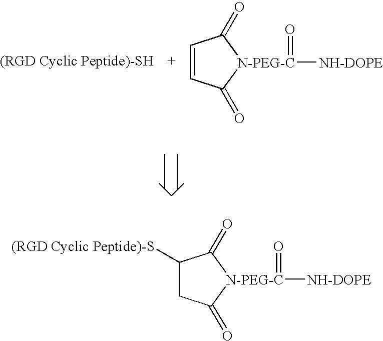

7. The active targeting liposome of claim 6, wherein the membrane material of the liposome is composed of egg phospholipid, cholesterol, monooxymethyl-polyethylene glycol lipid derivative and maleoyl-polyethylene glycol lipid derivative.

8. The active targeting liposome of claim 7, wherein the molar ratio of the membrane material is egg phospholipid:cholesterol:monooxymethyl-polyethylene glycol lipid derivatives:maleoyl-polyethylene glycol lipid derivatives=2:1:0.1:0.02.

9. The active targeting liposome of claim 7, wherein the content of polyethylene glycol in the membrane material is 3.2mol %.

10. The active targeting liposome of claim 6, wherein the active targeting liposome is loaded with interferon to form a drug-loaded active targeting liposome.

11. The active targeting liposome of claim 6, wherein the free hydrosulfide group of cysteine residue of the RGD cyclic peptide is the conjugation site with the liposome.

12. The active targeting liposome of claim 6, wherein the particle size of the liposome ranges between 50˜200 nm.

13. The active targeting liposome of claim 6, wherein the particle size of the liposome is 100 nm.

14. The active targeting liposome of claim 10, wherein said active targeting liposome is used in preparation of drugs for treatment of hepatic fibrosis.

15. The active targeting liposome of claim 10, wherein said active targeting liposome is used in preparation of drugs for treatment of hepatic fibrosis for intravenous use.
